(19) United States
(12) Patent Application Publication
(10) Pub. No.: US 2022/0039692 A1
(43) Pub. Date: Feb. 10, 2022

(54) METHOD AND PROCESSING DEVICE FOR ASSESSING VOLUME RESPONSIVENESS

(71) Applicant: SHENZHEN DARMA TECHNOLOGY CO., LTD., Shenzhen City, Guangdong Province (CN)

(72) Inventor: SHAOCHUN ZHUANG, Shenzhen City, Guangdong Province (CN)

(21) Appl. No.: 17/275,649

(22) PCT Filed: Sep. 11, 2018
(86) PCT No.: PCT/CN2018/105065

§ 371 Date: Mar. 11, 2021

(51) Int. Cl. A61B 5/11; A61B 5/00; A61B 5/349

(52) U.S. Cl. CPC A61B 5/11; A61B 5/7203; A61B 2562/0266; A61B 2562/0219; A61B 2562/0247; A61B 5/349

(57) ABSTRACT

The present invention belongs to the field of medicine and discloses a method for assessing volume responsiveness and a processing device for assessing volume responsiveness. The method comprises: acquiring, by using one or more vibration sensitive sensors, a first parameter associated with a change in preload in a first time interval before a subject performs a passive straight-leg lift in a passive leg raising (PLR) test; acquiring, by using the one or more vibration sensitive sensors, a second parameter associated with a change in preload in a second time interval after the subject performs a passive straight-leg lift in the PLR test; and determining the volume responsiveness of the subject according to the first parameter associated with a change in preload and the second parameter associated with a change in preload. The method provides a convenient and easy determination of volume responsiveness.

### FIELD OF THE INVENTION

The present invention belongs to the field of medicine, and particularly relates to a method and a processing device for assessing volume responsiveness.

### BACKGROUND OF THE INVENTION

Volume management is one of the important topics of ICU (Intensive Care Unit) and CCU (Cardiac Care Unit). Volume responsiveness assessment mainly evaluates the preload reserve, that is, whether cardiac output increases when preload increases.

Invasive methods for assessing volume responsiveness will cause inconvenience to the patient.

### Technical Problem

The present invention provides a method, a device, a system, a computer-readable storage medium, and a processing device for assessing volume responsiveness, and aims to solve the problem of inconvenience caused by invasive methods to patients.

### Technical Solutions

In the first aspect, the present invention provides a method for assessing volume responsiveness, comprising:

acquiring, by means of one or more vibration sensors, a first parameter associated with a change in preload in a first time interval before a subject performs a Passive Straight-Leg Lift in a Passive Leg Raising (PLR) test;

acquiring, by means of the one or more vibration sensors, a second parameter associated with a change in preload in a second time interval after the subject performs a Passive Straight-Leg Lift in the PLR test; and

determining the volume responsiveness of the subject according to the first parameter associated with a change in preload and the second parameter associated with a change in preload.

In the second aspect, the present invention provides a device for assessing volume responsiveness, comprising:

a first parameter acquisition unit, used for acquiring, by means of one or more vibration sensors, a first parameter associated with a change in preload in a first time interval before a subject performs a Passive Straight-Leg Lift in a Passive Leg Raising (PLR) test;

a second parameter acquisition unit, used for acquiring, by means of the one or more vibration sensors, a second parameter associated with a change in preload in a second time interval after the subject performs a Passive Straight-Leg Lift in the PLR test; and

a volume responsiveness determination unit, used for determining the volume responsiveness of the subject according to the first parameter associated with a change in preload and the second parameter associated with a change in preload.

In a third aspect, the present invention provides a computer-readable storage medium that stores computer programs that, when executed by processors, implement the steps of the method for assessing volume responsiveness as described above.

In a fourth aspect, the present invention provides a processing device for assessing volume responsiveness, comprising:

one or more processors;

a memory; and

one or more computer programs, wherein the one or more computer programs are stored in the memory and are configured to be executed by the one or more processors, and when executed by the one or more processors, implement the steps of the method for assessing volume responsiveness as described above.

In a fifth aspect, the present invention provides a system for assessing volume responsiveness, comprising:

one or more vibration sensors, configured to be placed in a predetermined position to acquire vibration information of the subject; and

the processing device for assessing volume responsiveness as the above mentioned, and being connected with the one or more vibration sensors.

### Advantages

In the present invention, acquiring, by means of one or more vibration sensors, a first parameter associated with a change in preload in a first time interval before a subject performs a Passive Straight-Leg Lift and a change in preload in a second time interval after the subject performs a Passive Straight-Leg Lift in a Passive Leg Raising (PLR) test; and determining the volume responsiveness of the subject according to the first parameter associated with a change in preload and the second parameter associated with a change in preload; therefore, the method provides a convenient and easy determination of volume responsiveness, and a standard deviation of the method provided in the present invention can be as small as about 4 ms.

### BRIEF DESCRIPTION OF THE DRAWINGS

FIG. 1 is a flow chart of a method for assessing volume responsiveness in accordance with a first embodiment of the present invention;

FIG. 2 is a functional block diagram of a device for assessing volume responsiveness in accordance with a second embodiment of the present invention;

FIG. 3 is a specific structural block diagram of a processing device for assessing volume responsiveness in accordance with a fourth embodiment of the present invention; and

FIG. 4 is a specific structural block diagram of a system for assessing volume responsiveness in accordance with a fifth embodiment of the present invention.

### BEST MODE OF THE INVENTION

In order to make the objectives, technical solutions, and advantages of the present invention clearer, the following further describes the present invention in detail with reference to the accompanying drawings and embodiments. It should be understood that the specific embodiments described here are only used to explain the present invention, but not to limit the present invention.

In order to illustrate the technical solutions of the present invention, the following is illustrated by specific embodiments.

### Description of Professional Terms

EMD: electrical mechanical delay

MPI: myocardial performance Index

SPI: Systole Performance Index

IVCT: isovolumetric contraction time

IVRT: isovolumetric relaxation time

LVET: Left Ventricle Eject Time

MVC/MC: mitral valve closure

AVO: Aortic valve opening

AVC: Aortic valve closure

MVO/MO: Mitral valve opening

SV: stroke volume

PLR: Passive Leg Rising, Preload: Preload

Afterload: Afterload

PEP: pre-ejection period

### First Embodiment

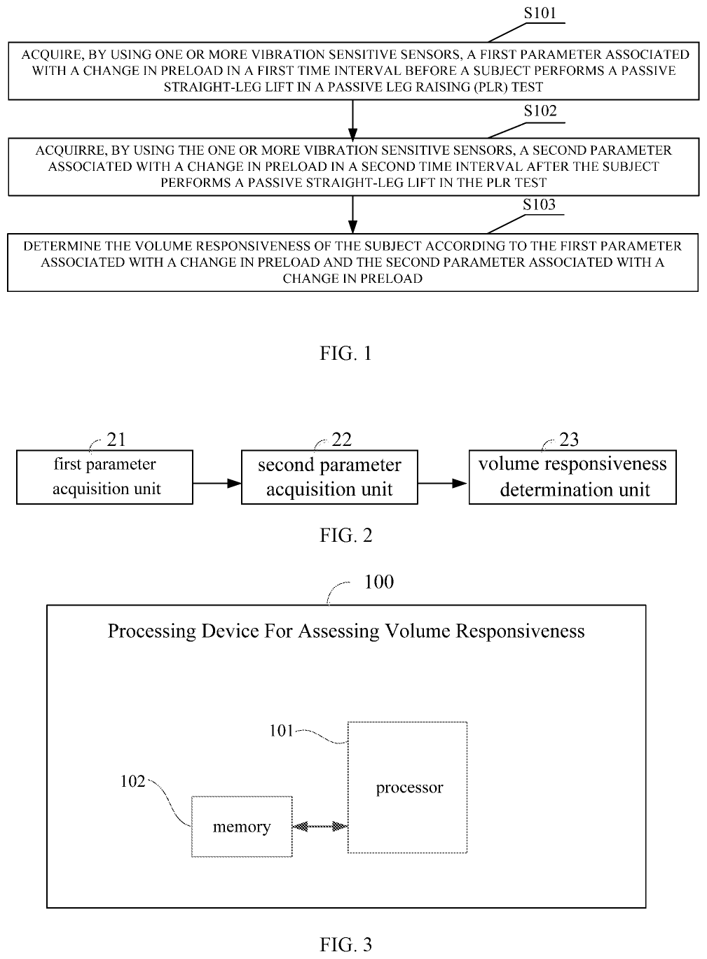

Referring to FIG. 1, a method for assessing volume responsiveness provided in the first embodiment, comprises the following steps of: it should be noted that if there are substantially the same results, the method for assessing volume responsiveness of the present invention is not limited to the process sequence shown in FIG. 1.

S101: acquiring, by means of one or more vibration sensors, a first parameter associated with a change in preload in a first time interval before a subject performs a Passive Straight-Leg Lift in a Passive Leg Raising (PLR) test.

S102: acquiring, by means of the one or more vibration sensors, a second parameter associated with a change in preload in a second time interval after the subject performs a Passive Straight-Leg Lift in the PLR test.

PLR test refers to an assessment of the body's volume responsiveness by monitoring the changes in SV or other alternative indicators (such as peak aortic blood flow, pulse pressure, etc.) in a time interval before and after Passive Straight-Leg Lift. The steps are as follows: collecting data of a supine or semi-recumbent (for example, at 45 degrees) subject in the first time interval (the first time interval can include one or more breathing cycles); if the subject is in a semi-recumbent position in the first step, changes to a supine position, and then performs a passive straight-leg lift to 45 degrees; and collecting data of the subject in the second time interval (the second time period can include one or more breathing cycles).

S103: determining the volume responsiveness of the subject according to the first parameter associated with a change in preload and the second parameter associated with a change in preload.

In the first embodiment of the present invention, the vibration sensors may be one or more of: an acceleration sensor, a speed sensor, a displacement sensor, a pressure sensor, a strain sensor, a stress sensor, or sensors (such as electrostatic sensors, inflatable micro-motion sensors, radar sensors, etc.) that convert physical quantities equivalently on the basis of acceleration, speed, displacement, or pressure. The strain sensor may be a fiber-optic sensor. The vibration sensor can be configured to be placed on various types of beds such as medical beds and nursing beds where the subject is located. The subject may be a living body for vital signal monitoring. In some embodiments, the subject may be a hospital patient or a person being cared for, such as an elderly person, an imprisoned person, or other people.

The fiber-optic strain sensor comprises:

an optical fiber, disposed substantially in one plane;

a light source, coupled to one end of the optical fiber;

a receiver, coupled to the other end of the optical fiber, and configured to sense changes in intensity of light transmitted through the optical fiber; and

a mesh layer, composed of meshes with openings; the mesh layer is in contact with the surface of the optical fiber.

In the first embodiment of the present invention, S101 may specifically comprises the following steps of.

S1011: acquiring first vibration information of a supine or semi-recumbent subject in the first time interval by means of the one or more vibration sensors.

In the first embodiment of the present invention, when the vibration sensor is a speed sensor, a displacement sensor, a pressure sensor, a strain sensor, a stress sensor, or a sensor that convert physical quantities equivalently on the basis of acceleration, speed, displacement, or pressure, the one or more vibration sensors may be configured to be placed under the shoulders and/or the back of the supine or semi-recumbent subject; when the vibration sensor is an acceleration sensor, the acceleration sensor is configured to be placed on the body section above the subject's sternum.

S1012: generating first hemodynamic related information on the basis of the first vibration information.

In the first embodiment of the present invention, S1012 may specifically be:

preprocessing the first vibration information to generate the first hemodynamic related information; wherein the preprocessing comprises at least one of: filtering, denoising, and signal scaling.

S1013: acquiring the first parameter associated with a change in preload in the first time interval on the basis of the first hemodynamic related information.

In the first embodiment of the present invention, S102 may specifically comprise the following steps of:

S1021: acquiring, by means of the one or more vibration sensors, second vibration information of the subject in the second time interval after the supine subject performs a Passive Straight-Leg Lift in the PLR test.

In the first embodiment of the present invention, the one or more vibration sensors may be configured to be placed under the shoulders and/or the back of the supine subject. When the subject to be measured is in a resting state, the second vibration information acquired by the vibration sensors comprises at least one of: vibration information caused by breathing, vibration information caused by contraction and relaxation of the heart, human body movement information, and body vibration information caused by blood vessel wall deformation.

S1022: generating second hemodynamic related information on the basis of the second vibration information.

In the first embodiment of the present invention, S1022 may specifically be:

preprocessing the second vibration information to generate the second hemodynamic related information; wherein the preprocessing comprises at least one of: filtering, denoising, and signal scaling.

S1023: acquiring the second parameter associated with a change in preload in the second time interval on the basis of the second hemodynamic related information.

In the first embodiment of the present invention, when the first parameter associated with a change in preload is IVCT, LVET and SPI, S1011 can specifically be:

acquiring the first vibration information of the supine or semi-recumbent subject in the first time interval by means of a vibration sensor configured to be placed under the subject's left or right shoulder.

S1013 can specifically be:

identifying MC time point, AVO time point, and AVC time point in each cardiac cycle in the first time interval from the first hemodynamic related information, where the first time interval comprises at least one breathing cycle; specifically can comprise the following steps: extracting high-frequency component from the first hemodynamic related information, for example, performing high-frequency component extraction by polynomial fitting and smoothing filter method; when the vibration sensor is a fiber-optic sensor, performing the second-order differential operation on the first hemodynamic related information when extracting high-frequency component from the first hemodynamic related information; and performing feature search on the first hemodynamic related information after the second-order differential operation to determine the MC time point, AVO time point and AVC time point in each cardiac cycle in the first time interval; when the vibration sensor is an acceleration sensor, directly performing feature search on the first hemodynamic related information to determine the MC time point, AVO time point and AVC time point in each cardiac cycle in the first time interval; and

obtaining the IVCT, LVET and SPI in the first time interval according to the MC time point, AVO time point and AVC time point in each cardiac cycle; specifically: taking a breathing cycle as a data processing interval, averaging the IVCT, LVET and SPI in each cardiac cycle in this breathing cycle as the value of IVCT, LVET and SPI in this breathing cycle; and calculating to obtain a mean IVCT value, a mean LVET value, and a mean SPI value in the first time interval according to the value of IVCT, LVET and SPI in each breathing cycle.

When the second parameter associated with a change in preload is IVCT, LVET, and SPI, S1021 may specifically be:

acquiring, by means of a vibration sensor configured to be placed under the subject's left or right shoulder, the second vibration information of the subject in the second time interval after the supine subject performs a Passive Straight-Leg Lift in the PLR test.

S1023 can specifically be:

identifying MC time point, AVO time point, and AVC time point in each cardiac cycle in the second time interval from the second hemodynamic related information, where the second time interval comprises at least one breathing cycle; specifically can comprise the following steps: extracting high-frequency component from the second hemodynamic related information, for example, performing high-frequency component extraction by polynomial fitting and smoothing filter method; when the vibration sensor is a fiber-optic sensor, performing the second-order differential operation on the second hemodynamic related information when extracting high-frequency component from the second hemodynamic related information; and performing feature search on the second hemodynamic related information after the second-order differential operation to determine the MC time point, AVO time point and AVC time point in each cardiac cycle in the second time interval; when the vibration sensor is an acceleration sensor, and when extracting high-frequency component from the second hemodynamic related information, directly performing feature search on the second hemodynamic related information to determine the MC time point, AVO time point and AVC time point in each cardiac cycle in the second time interval and

obtaining the IVCT, LVET and SPI in the second time interval according to the MC time point, AVO time point and AVC time point in each cardiac cycle; specifically: taking a breathing cycle as a data processing interval, averaging the IVCT, LVET and SPI in each cardiac cycle in this breathing cycle as the value of IVCT, LVET and SPI in this breathing cycle; and calculating to obtain a mean IVCT value, a mean LVET value, and a mean SPI value in the second time interval according to the value of IVCT, LVET and SPI in each breathing cycle.

In the first embodiment of the present invention, when the first parameter associated with a change in preload is IVCT, LVET and SPI, S1011 can specifically be:

acquiring the left shoulder first vibration information of the supine or semi-recumbent subject in the first time interval by means of a vibration sensor configured to be placed under the subject's left shoulder; and acquiring the right shoulder first vibration information of the supine or semi-recumbent subject in the first time interval by means of a vibration sensor configured to be placed under the subject's right shoulder.

S1012 may also be specifically:

generating the left shoulder first hemodynamic related information according to the left shoulder first vibration information, and generating the right shoulder first hemodynamic related information according to the right shoulder first vibration information.

S1013 may also be specifically:

identifying the MC time point and the AVO time point in each cardiac cycle in the first time interval from the left shoulder first hemodynamic related information, and identifying the AVC time point in each cardiac cycle in the first time interval from the right shoulder first hemodynamic related information, wherein the first time interval comprises at least one breathing cycle;

obtaining the IVCT, LVET and SPI in the first time interval according to the MC time point, AVO time point and AVC time point in each cardiac cycle; specifically: taking a breathing cycle as a data processing interval, averaging the IVCT, LVET and SPI in each cardiac cycle in this breathing cycle as the value of IVCT, LVET and SPI in this breathing cycle; and calculating to obtain a mean IVCT value, a mean LVET value, and a mean SPI value in the first time interval according to the value of IVCT, LVET and SPI in each breathing cycle.

When the second parameter associated with a change in preload is IVCT, LVET, and SPI, S1021 may specifically be:

acquiring the left shoulder second vibration information of the supine subject in the second time interval, by means of a vibration sensor configured to be placed under the subject's left shoulder; and acquiring the right shoulder second vibration information of the supine subject in the second time interval, by means of a vibration sensor configured to be placed under the subject's right shoulder.

S1022 may specifically be:

generating the left shoulder second hemodynamic related information on the basis of the left shoulder second vibration information, and generating the right shoulder second hemodynamic related information on the basis of the righter shoulder second vibration information.

S1023 may also be specifically:

identifying the MC time point and the AVO time point in each cardiac cycle in the second time interval from the left shoulder second hemodynamic related information, and identifying the AVC time point in each cardiac cycle in the second time interval from the right shoulder second hemodynamic related information, wherein the second time interval comprises at least one breathing cycle; and

obtaining the IVCT, LVET and SPI in the second time interval according to the MC time point, AVO time point and AVC time point in each cardiac cycle; specifically: taking a breathing cycle as a data processing interval, averaging the IVCT, LVET and SPI in each cardiac cycle in this breathing cycle as the value of IVCT, LVET and SPI in this breathing cycle; and calculating to obtain a mean IVCT value, a mean LVET value, and a mean SPI value in the second time interval according to the value of IVCT, LVET and SPI in each breathing cycle.

In the embodiment of the acceleration sensor, the acceleration sensor is placed on the body section above the sternum of the subject. The subject can lie flat or stand in a resting state. At this time, the acceleration sensor needs to be fixed on the body section corresponding to the sternum using medical tape, gel, etc., or something like a strap. From top to bottom, the human sternum is the manubrium, the body of the sternum and the xiphoid process. Preferably, the acceleration sensor is placed on the body section corresponding to the body of the sternum, and more preferably, the acceleration sensor is placed on the body section corresponding to the lower end of the body of the sternum, that is, the body part on one side of the xiphoid process.

S103 may specifically be:

calculating a SPI difference between the mean SPI value in the first time interval and the mean SPI value in the second time interval; wherein: if the SPI difference is in a first interval, then judging the volume responsiveness of the subject to be positive, and vice versa, judging the volume responsiveness of the subject to be negative; for example, the mean SPI value in the first time interval SPI=IVCT/LVET=47/175=0.269, and the mean SPI value in the second time interval SPI=IVCT/LVET=38/250=0.152, judging the volume responsiveness of the subject to be positive, that is, the SPI difference exceeds 0.1, and the volume responsiveness of the subject is considered positive; or,

calculating an IVCT difference between the mean IVCT value in the first time interval and the mean IVCT value in the second time interval; wherein: if the IVCT difference is in a second interval, then judging the volume responsiveness of the subject to be positive, and vice versa, judging the volume responsiveness of the subject to be negative; for example, the mean IVCT value before raising the leg is 47, and the mean IVCT value after raising the leg is 38; the difference is greater than 6 ms, the subject's volume responsiveness is considered positive; or,

calculating an LVET difference between the mean LVET value in the first time interval and the mean LVET value in the second time interval; wherein: if the LVET difference is in a third interval, judging the volume responsiveness of the subject to be positive, and vice versa, judging the volume responsiveness of the subject to be negative; for example, if the LVET difference between before and after exceeds 10%, the subject's volume responsiveness is considered positive; or,

calculating a PEP difference between the mean PEP value in the first time interval and the mean PEP value in the second time interval; wherein: if the PEP difference is in a fourth interval, judging the volume responsiveness of the subject to be positive, and vice versa, judging the volume responsiveness of the subject to be negative; for example, PEP=IVCT+EMD, if the PEP difference between before and after exceeds 15 ms, the subject's volume responsiveness is considered positive.

In the first embodiment of the present invention, when the first parameter associated with a change in preload is EMD, S1011 can specifically be:

acquiring the first vibration information of the supine or semi-recumbent subject in the first time interval by means of a vibration sensor configured to be placed under the subject's left shoulder.

S1013 can specifically be:

identifying the MC time point in each cardiac cycle in the first time interval from the first hemodynamic related information, where the first time interval comprises at least one breathing cycle; specifically can comprise the following steps: extracting high-frequency component from the first hemodynamic related information, for example, performing high-frequency component extraction by polynomial fitting and smoothing filter method: when the vibration sensor is a fiber-optic sensor, performing the fourth-order differential operation on the first hemodynamic related information when extracting high-frequency component from the first hemodynamic related information; and performing feature search on the first hemodynamic related information after the fourth-order differential operation to determine the MC time point in each cardiac cycle in the first time interval;

acquiring an electrocardiogram (ECG) signal of the subject through an ECG data acquisition device; and

calculating the EMD in the first time interval on the basis of the first hemodynamic related information and the ECG signal of the subject, where the starting point of the EMD is the time point corresponding to the Q wave of the ECG signal, and the end point is the MC time point of the first hemodynamic related information.

When the second parameter associated with a change in preload is EMD, S1021 may specifically be:

acquiring, by means of a vibration sensor configured to be placed under the subject's left shoulder, the second vibration information of the subject in the second time interval after the supine subject performs a Passive Straight-Leg Lift in the PLR test.

S1023 can specifically be:

identifying the MC time point in each cardiac cycle in the second time interval from the second hemodynamic related information, where the second time interval comprises at least one breathing cycle; specifically can comprise the following steps: extracting high-frequency component from the second hemodynamic related information, for example, performing high-frequency component extraction by polynomial fitting and smoothing filter method; when the vibration sensor is a fiber-optic sensor, performing the fourth-order differential operation on the second hemodynamic related information when extracting high-frequency component from the second hemodynamic related information; and performing feature search on the second hemodynamic related information after the fourth-order differential operation to determine the MC time point, in each cardiac cycle in the second time interval;

acquiring an ECG signal of the subject through an ECG data acquisition device; and

calculating the EMD in the second time interval on the basis of the second hemodynamic related information and the ECG signal of the subject, where the starting point of the EMD is the time point corresponding to the Q wave of the ECG signal, and the end point is the MC time point of the second hemodynamic related information.

S103 may specifically be:

calculating an EMD difference between the EMD in the first time interval and the EMD in the second time interval; wherein: if the EMD in the second time interval is smaller than the EMD in the first time interval, and the EMD difference is within a preset value range, then judging the volume responsiveness of the subject to be positive, and vice versa, judging the volume responsiveness of the subject to be negative.

In the first embodiment of the present invention, the volume responsiveness of the subject can also be judged jointly based on the changes of the SPI and EMD. When both SPI reduction and EMD reduction are satisfied, judge the volume responsiveness of the subject to be positive.

The volume responsiveness of the subject can also be judged on the basis of PEP, where PEP=IVCT+EMD, and if the PEP difference before and after exceeds 15 ms, the volume responsiveness of the subject is considered positive.

In the first embodiment of the present invention, when the first parameter and the second parameter associated with a change in preload are PEP, S1011 can specifically be:

acquiring the first vibration information of the supine or semi-recumbent subject in the first time interval by means of a vibration sensor configured to be placed under the subject's left shoulder.

S1013 can specifically be:

identifying MC time point, AVO time point and AVC time point in each cardiac cycle in the first time interval from the first hemodynamic related information;

acquiring an ECG signal of the subject through an ECG data acquisition device; and

calculating the EMD in the first time interval on the basis of the first hemodynamic related information and the ECG signal of the subject, obtaining IVCT in the first time interval according to the MC time point, AVO time point and AVC time point in each cardiac cycle; obtaining PEP by adding IVCT and EMD; where the starting point of the EMD is the time point corresponding to the Q wave of the ECG signal, and the end point is the MC time point of the first hemodynamic related information.

S1021 may specifically be:

acquiring, by means of a vibration sensor configured to be placed under the subject's left shoulder, the second vibration information of the subject in the second time interval after the supine subject performs a Passive Straight-Leg Lift in the PLR test.

S1023 may specifically comprise:

identifying MC time point, AVO time point, and AVC time point in each cardiac cycle in the second time interval from the second hemodynamic related information;

acquiring the ECG signal of the subject through an ECG data acquisition device;

calculating the EMD in the second time interval on the basis of the second hemodynamic related information and the ECG signal of the subject, obtaining IVCT in the second time interval according to the MC time point, AVO time point and AVC time point in each cardiac cycle; obtaining PEP by adding IVCT and EMD; where the starting point of the EMD is the time point corresponding to the Q wave of the ECG signal, and the end point is the MC time point of the second hemodynamic related information.

The S103 may specifically be:

calculating a PEP difference between the mean PEP value in the first time interval and the mean PEP value in the second time interval; wherein: if the PEP difference is in a fourth interval, judging the volume responsiveness of the subject to be positive, and vice versa, judging the volume responsiveness of the subject to be negative.

### Second Embodiment

Referring to FIG. 2, a device for assessing volume responsiveness in the second embodiment of the present invention, comprises:

a first parameter acquisition unit 21, used for acquiring, by means of one or more vibration sensors, a first parameter associated with a change in preload in a first time interval before a subject performs a Passive Straight-Leg Lift in a Passive Leg Raising (PLR) test;

a second parameter acquisition unit 22, used for acquiring, by means of the one or more vibration sensors, a second parameter associated with a change in preload in a second time interval after the subject performs a Passive Straight-Leg Lift in the PLR test; and

a volume responsiveness determination unit 23, used for determining the volume responsiveness of the subject according to the first parameter associated with a change in preload and the second parameter associated with a change in preload.

The device for assessing volume responsiveness provided in the second embodiment of the present invention and the method for assessing volume responsiveness provided in the first embodiment of the present invention belong to the same concept, and the specific implementation process is detailed in the full text of the description, and will not be repeated here.

### Third Embodiment

The third embodiment of the present invention provides a computer-readable storage medium that stores computer programs that, when executed by processors, implement the steps of the method for assessing volume responsiveness provided in the first embodiment of the present invention.

### Fourth Embodiment

FIG. 3 shows a specific structural block diagram of a processing device for assessing volume responsiveness provided in the fourth embodiment of the present invention. The processing device 100 for assessing volume responsiveness comprises: one or more processors 101, a memory 102, and one or more computer programs, wherein the one or more processors 101 and the memory 102 are connected by a bus. The one or more computer programs are stored in the memory 102 and are configured to be executed by the one or more processors 101, and when executed by the one or more processors, implement the steps of the method for assessing volume responsiveness provided in the first embodiment of the present invention.

### Fifth Embodiment

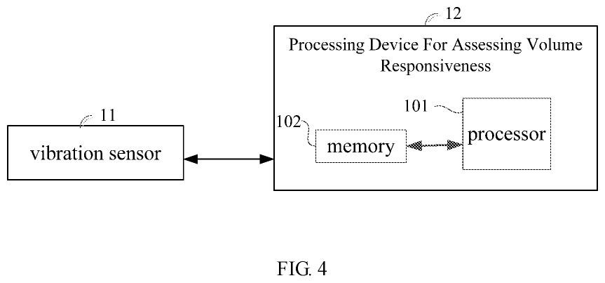

Referring to FIG. 4, a system for assessing volume responsiveness provided by the fifth embodiment of the present invention, comprises:

one or more vibration sensors 11, configured to be placed in a predetermined position to acquire vibration information of the subject; and

the processing device 12 for assessing volume responsiveness provided in the fourth embodiment of the present invention, and being connected with the one or more vibration sensors.

The system for assessing volume responsiveness provided by the fifth embodiment of the present invention, may further comprise: an ECG data acquisition device for acquiring the ECG signal of the subject.

The system for assessing volume responsiveness may further comprise:

an output device connected to the processing device for assessing volume responsiveness and/or the vibration sensors. The vibration sensor transmits the acquired vibration information to the output device for output, and the processing device for assessing volume responsiveness transmits the processed result to the output device for output.

The system for assessing volume responsiveness may further comprise: an input device (such as a mouse, a keyboard) for user input so that the processing device for assessing volume responsiveness determines MC time point, AVO time point, and AVC time point according to user input.

In the present invention, acquiring, by means of one or more vibration sensors, a first parameter associated with a change in preload in a first time interval before a subject performs a Passive Straight-Leg Lift and a change in preload in a second time interval after the subject performs a Passive Straight-Leg Lift in a Passive Leg Raising (PLR) test; and determining the volume responsiveness of the subject according to the first parameter associated with a change in preload and the second parameter associated with a change in preload; therefore, the method provides a convenient and easy determination of volume responsiveness, and a standard deviation of the method provided in the present invention can be as small as about 4 ms.

Those of ordinary skill in the art can understand that all or part of the steps in the various methods of the above-mentioned embodiments can be completed by a program instructing relevant hardware, and the program can be stored in a computer-readable storage medium. It may include: Read Only Memory (ROM, Read Only Memory), Random Access Memory (RAM), magnetic disk or optical disk, etc.

The foregoing descriptions are only preferred embodiments of the present invention, and are not intended to limit the present invention. Any modification, equivalent replacement and improvement made within the spirit and principle of the present invention shall be included within the scope of protection of the invention.

1. A method for assessing volume responsiveness, comprising steps of:
S101, acquiring, by means of one or more vibration sensors, a first parameter associated with a change in preload in a first time interval before a subject performs a Passive Straight-Leg Lift in a Passive Leg Raising PLR test;
S102, acquiring, by means of the one or more vibration sensors, a second parameter associated with a change in preload in a second time interval after the subject performs a Passive Straight-Leg Lift in the PLR test; and
S103, determining the volume responsiveness of the subject according to the first parameter associated with a change in preload and the second parameter associated with a change in preload.

2. The method of claim 1, wherein the first time interval comprises at least one breathing cycle; and the second time interval comprises at least one breathing cycle.

3. The method of claim 1, wherein the vibration sensor is selected from one or more of: an acceleration sensor, a speed sensor, a displacement sensor, a pressure sensor, a strain sensor, a stress sensor, or sensors that convert physical quantities equivalently on the basis of acceleration, speed, pressure, or displacement.

4. The method of claim 3, wherein the strain sensor is a fiber-optic sensor;
fiber-optic sensor comprises:
an optical fiber, disposed substantially in one plane;
a light source, coupled to one end of the optical fiber;
a receiver, coupled to the other end of the optical fiber, and configured to sense changes in intensity of light transmitted through the optical fiber; and
a mesh layer, composed of meshes with openings, and being in contact with a surface of the optical fiber.

5. The method of claim 3, wherein S101 specifically comprises:
S1011, acquiring first vibration information of a supine or semi-recumbent subject in the first time interval by means of the one or more vibration sensors;
S1012, generating first hemodynamic related information on the basis of the first vibration information; and
S1013, acquiring the first parameter associated with a change in preload in the first time interval on the basis of the first hemodynamic related information.

6. The method of claim 5, wherein S102 specifically comprises:
S1021, acquiring, by means of the one or more vibration sensors, second vibration information of the subject in the second time interval after the supine subject performs a Passive Straight-Leg Lift in the PLR test;
S1022, generating second hemodynamic related information on the basis of the second vibration information; and
S1023, acquiring the second parameter associated with a change in preload in the second time interval on the basis of the second hemodynamic related information.

7. The method of claim 6, wherein the one or more vibration sensors are configured to be placed under the shoulder and/or the back of the subject.

8. The method of claim 6, wherein when the vibration sensor is an acceleration sensor, the acceleration sensor is configured to be placed on the body section above the subject's sternum.

9. The method of claim 6, wherein S1012 specifically is:
preprocessing the first vibration information to generate the first hemodynamic related information;
S1022 specifically is:
preprocessing the second vibration information to generate the second hemodynamic related information;
wherein the preprocessing comprises at least one of: filtering, denoising, and signal scaling.

10. The method of claim 6, wherein when the first parameter and the second parameter associated with a change in preload are IVCT LVET and SPI;
S1013 specifically comprises:
identifying MC time point, AVO time point, and AVC time point in each cardiac cycle in the first time interval from the first hemodynamic related information; and
obtaining the IVCT, LVET and SPI in the first time interval according to the MC time point, AVO time point and AVC time point in each cardiac cycle;
S1023 specifically comprises:
identifying MC time point, AVO time point, and AVC time point in each cardiac cycle in the second time interval from the second hemodynamic related information; and
obtaining the IVCT, LVET and SPI in the second time interval according to the MC time point, AVO time point and AVC time point in each cardiac cycle.

11. The method of claim 10, wherein the step of “identifying MC time point, AVO time point, and AVC time point in each cardiac cycle in the first time interval from the first hemodynamic related information”, specifically comprises the following steps of:
extracting high-frequency component from the first hemodynamic related information, when the vibration sensor is a fiber-optic sensor, performing the second-order differential operation on the first hemodynamic related information when extracting high-frequency component from the first hemodynamic related information; and performing feature search on the first hemodynamic related information after the second-order differential operation to determine the MC time point, AVO time point and AVC time point in each cardiac cycle in the first time interval; when the vibration sensor is an acceleration sensor, directly performing feature search on the first hemodynamic related information to determine the MC time point, AVO time point and AVC time point in each cardiac cycle in the first time interval;
the step of “obtaining the IVCT, LVET and SPI in the first time interval according to the MC time point, AVO time point and AVC time point in each cardiac cycle”, specifically is:
averaging the IVCT, LVET and SPI in each cardiac cycle in the first time interval to obtain a mean IVCT value, a mean LVET value and a mean SPI value as the value of IVCT, LVET and SPI in the first time interval;
the step of “identifying MC time point, AVO time point, and AVC time point in each cardiac cycle in the second time interval from the second hemodynamic related information” specifically comprises the following steps of:
extracting high-frequency component from the second hemodynamic related information, when the vibration sensor is a fiber-optic sensor, performing the second-order differential operation on the second hemodynamic related information when extracting high-frequency component from the second hemodynamic related information; and performing feature search on the second hemodynamic related information after the second-order differential operation to determine the MC time point, AVO time point and AVC time point in each cardiac cycle in the second time interval; when the vibration sensor is an acceleration sensor, directly performing feature search on the second hemodynamic related information to determine the MC time point, AVO time point and AVC time point in each cardiac cycle in the second time interval;
the step of “obtaining the IVCT, LVET and SPI in the second time interval according to the MC time point, AVO time point and AVC time point in each cardiac cycle” specifically is:
averaging the IVCT, LVET and SPI in each cardiac cycle in the second time interval to obtain a mean IVCT value, a mean LVET value and a mean SPI value as the value of IVCT, LVET and SPI in the second time interval.

12. The method of claim 10, wherein S103 specifically is:
calculating a SPI difference between the mean SPI value in the first time interval and the mean SPI value in the second time interval; wherein: if the SPI difference is in a first interval, then judging the volume responsiveness of the subject to be positive, and vice versa, judging the volume responsiveness of the subject to be negative; or
calculating an IVCT difference between the mean IVCT value in the first time interval and the mean IVCT value in the second time interval; wherein: if the IVCT difference is in a second interval, then judging the volume responsiveness of the subject to be positive, and vice versa, judging the volume responsiveness of the subject to be negative; or
calculating an LVET difference between the mean LVET value in the first time interval and the mean LVET value in the second time interval; wherein: if the LVET difference is in a third interval, judging the volume responsiveness of the subject to be positive, and vice versa, judging the volume responsiveness of the subject to be negative; or
calculating a PEP difference between the mean PEP value in the first time interval and the mean PEP value in the second time interval; wherein: if the PEP difference is in a fourth interval, judging the volume responsiveness of the subject to be positive, and vice versa, judging the volume responsiveness of the subject to be negative.

13. The method of claim 6, wherein when the first parameter and the second parameter associated with a change in preload are EMD;
S1011 specifically is:
acquiring the first vibration information of the supine or semi-recumbent subject in the first time interval by means of a vibration sensor configured to be placed under the subject's left shoulder;
S1013 specifically is:
identifying the MC time point in each cardiac cycle in the first time interval from the first hemodynamic related information;
acquiring an electrocardiogram ECG signal of the subject through an ECG data acquisition device; and
calculating the EMD in the first time interval on the basis of the first hemodynamic related information and the ECG signal of the subject, where a starting point of the EMD is a time point corresponding to the Q wave of the ECG signal, and an endpoint is the MC time point of the first hemodynamic related information;
S1021 specifically is:
acquiring, by means of a vibration sensor configured to be placed under the subject's left shoulder, the second vibration information of the subject in the second time interval after the supine subject performs a Passive Straight-Leg Lift in the PLR test;
S1023 specifically comprises:
identifying the MC time point in each cardiac cycle in the second time interval from the second hemodynamic related information;
acquiring an ECG signal of the subject through an ECG data acquisition device; and
calculating the EMD in the second time interval on the basis of the second hemodynamic related information and the ECG signal of the subject, where the starting point of the EMD is a time point corresponding to the Q wave of the ECG signal, and an end point is the MC time point of the second hemodynamic related information;
S103 specifically is:
calculating an EMD difference between the EMD in the first time interval and the EMD in the second time interval; wherein: if the EMD in the second time interval is smaller than the EMD in the first time interval, and the EMD difference is within a preset range, then judging the volume responsiveness of the subject to be positive, and vice versa, judging the volume responsiveness of the subject to be negative.

14. The method of claim 6, wherein when the first parameter and the second parameter associated with a change in preload are PEP;
S1011 specifically is:
acquiring the first vibration information of the supine or semi-recumbent subject in the first time interval by means of a vibration sensor configured to be placed under the subject's left shoulder;
S1013 specifically is:
identifying MC time point, AVO time point, and AVC time point in each cardiac cycle in the first time interval from the first hemodynamic related information;
acquiring an electrocardiogram ECG signal of the subject through an ECG data acquisition device; and
calculating the EMD in the first time interval on the basis of the first hemodynamic related information and the ECG signal of the subject, obtaining IVCT in the first time interval according to the MC time point, AVO time point and AVC time point in each cardiac cycle;
obtaining PEP by adding IVCT and EMD; where the starting point of the EMD is a time point corresponding to the Q wave of the ECG signal, and an end point is the MC time point of the first hemodynamic related information;
S1021 specifically is:
acquiring, by means of a vibration sensor configured to be placed under the subject's left shoulder, the second vibration information of the subject in the second time interval after the supine subject performs a Passive Straight-Leg Lift in the PLR test;
S1023 specifically comprises:
identifying the MC time point, AVO time point, and AVC time point in each cardiac cycle in the second time interval from the second hemodynamic related information;
acquiring an ECG signal of the subject through an ECG data acquisition device; and
calculating the EMD in the second time interval on the basis of the second hemodynamic related information and the ECG signal of the subject, obtaining IVCT in the second time interval according to the MC time point, AVO time point and AVC time point in each cardiac cycle; obtaining PEP by adding IVCT and EMD; where a starting point of the EMD is a time point corresponding to the Q wave of the ECG signal, and an end point is the MC time point of the second hemodynamic related information;
S103 specifically is:
calculating a PEP difference between the mean PEP value in the first time interval and the mean PEP value in the second time interval; wherein: if the PEP difference is in a fourth interval, judging the volume responsiveness of the subject to be positive, and vice versa, judging the volume responsiveness of the subject to be negative.

15. (canceled)

16. A non-transitory computer-readable storage medium that stores one or more computer programs that, when executed by one or more processors, implement the steps of the method for assessing volume responsiveness of claim 1.

17. A processing device for assessing volume responsiveness, comprising:
one or more processors;
a memory; and
one or more computer programs, wherein the one or more computer programs are stored in the memory and are configured to be executed by the one or more processors, and when executed by the one or more processors, implement a method for assessing volume responsiveness comprising steps of:
S101, acquiring, by means of one or more vibration sensors, a first parameter associated with a change in preload in a first time interval before a subject performs a Passive Straight-Leg Lift in a Passive Leg Raising PLR test;
S102, acquiring, by means of the one or more vibration sensors, a second parameter associated with a change in preload in a second time interval after the subject performs a Passive Straight-Leg Lift in the PLR test; and
S103, determining the volume responsiveness of the subject according to the first parameter associated with a change in preload and the second parameter associated with a change in preload.

18. A system for assessing volume responsiveness, comprising:
one or more vibration sensors, configured to be placed in a predetermined position to acquire vibration information of the subject; and
a processing device for assessing volume responsiveness being connected with the one or more vibration sensors, and comprising:
one or more processors;
a memory; and
one or more computer programs, wherein the one or more computer programs are stored in the memory and are configured to be executed by the one or more processors, and when executed by the one or more processors, implement a method for assessing volume responsiveness, comprising steps of:
S101, acquiring, by means of one or more vibration sensors, a first parameter associated with a change in preload in a first time interval before a subject performs a Passive Straight-Leg Lift in a Passive Leg Raising PLR test;
S102, acquiring, by means of the one or more vibration sensors, a second parameter associated with a change in preload in a second time interval after the subject performs a Passive Straight-Leg Lift in the PLR test; and
S103, determining the volume responsiveness of the subject according to the first parameter associated with a change in preload and the second parameter associated with a change in preload.

19. The system of claim 18, further comprising: an ECG data acquisition device for acquiring the ECG signal of the subject.

20. The system of claim 18, further comprising:
an output device connected to the processing device for assessing volume responsiveness and/or the one or more vibration sensors; wherein the one or more vibration sensors transmit the acquired vibration information to the output device for output, and the processing device for assessing volume responsiveness transmits the processed result to the output device for output.

21. The system of claim 18, further comprising: an input device, for user input so that the processing device for assessing volume responsiveness determines MC time point, AVO time point, and AVC time point according to user input.
